(19) United States
(12) Patent Application Publication
(10) Pub. No.: US 2016/0186248 A1
(43) Pub. Date: Jun. 30, 2016

(54) METHOD FOR CENTRIFUGE MOUNTABLE MANIFOLD FOR PROCESSING FLUIDIC ASSAYS

(71) Applicant: Tangen Biosciences, Inc., Branford, CT (US)

(72) Inventor: John Richard NOBILE, Guilford, CT (US)

(73) Assignee: Tangen Biosciences, Inc., Branford, CT (US)

(21) Appl. No.: 14/733,789

(22) Filed: Jun. 8, 2015

Related U.S. Application Data
(63) Continuation of application No. 14/650,529, filed on Jun. 8, 2015.
(60) Provisional application No. 61/737,751, filed on Dec. 15, 2012.

(51) Int. Cl. C12Q 1/68

(52) U.S. Cl. CPC C12Q 1/6844

(57) ABSTRACT

The disclosure generally relates to a method and apparatus for centrifuge mountable manifold for processing fluid assays. In one embodiment, the disclosure relates to a method for automatically running an assay with a radially configured flowrotor by: introducing a sample fluid to an inlet of the flowrotor; rotating the flowrotor in a first direction to provide a first radial acceleration to move the sample fluid from the inlet to a first reaction chamber, the first reaction chamber having one or more reactants; retaining the assay at the first reaction chamber for a first duration by maintain rotation at a first angular velocity to induce a reaction with the reactant and to provide a reacted assay; rapidly accelerating the flowrotor at a negative angular acceleration relative to the first angular velocity until the angular velocity is reversed thereby moving the reacted assay from the first reaction chamber to a second reaction chamber having an inlet and an outlet and containing a second reactant or reactants and retaining the assay at the second chamber for a second duration by continuing the second angular velocity to thereby induce a reaction with the second reactant.

The application claims priority to the filing date of PCT Application Serial No. PCT/US 13/75430 (filed Dec. 16. 2013) which claims priority to Provisional Patent Application No. 61/737,751 (filed Dec. 15, 2012) and to application Ser. No. 14/650,529 (filed Jun. 8, 2015); the specification of all cited applications are incorporated herein in entirety.

### BACKGROUND

1. Field

The disclosure relates to a method and apparatus for fluidic assay processing. Specifically, the disclosure relates to a method, apparatus and system for fluid manifold processor for automatic processing of a fluidic assay.

2. Description of Related Art

Isothermal nucleic acid amplification systems such as loop-mediated isothermal amplification (LOOP) are rapidly displacing conventional Polymerase Chain Reaction (PCR) type devices because they have similar sensitivity and specificity to PCR. However, isothermal amplification processes are faster and require less instrument complexity to implement in a diagnostic device. Conventional LAMP instruments are complex to use and thus require expert technicians working inside a lab, and they are also expensive. Because of this complexity and high potential for errors, such systems are not used at the point-of-care. Accordingly, there is a need for an automated instrument for LAMP-type systems that enable simple point of care detection.

### BRIEF DESCRIPTION OF THE DRAWINGS

These and other embodiments of the disclosure will be discussed with reference to the following exemplary and non-limiting illustrations, in which like elements are numbered similarly, and where:

FIG. 1 is a flowrotor having a single assay path according to one embodiment of the disclosure;

FIG. 2 is a plan view of the flowrotor part of FIG. 1;

FIG. 3 shows the fluid when introduced into the flowrotor;

FIG. 4 illustrates the fluid location after the rotor spins for the first time in the CCW direction;

FIG. 5 illustrate fluid relocation by reversing rotation direction;

FIG. 6 shows the fluid location after a second reversal of rotation direction from CW to CCW;

FIG. 7 shows the fluid location after the third reversal of rotation direction from CCW to CW;

FIG. 8 shows a multi-channel flowrotor according to one embodiment of the disclosure;

FIG. 9 illustrates an exemplary embodiment having bifurcated flow path;

FIGS. 10-20 show various fluid movement phases through the flowrotor according of FIG. 9;

FIG. 21 shows an exemplary flowrotor having two filling ports and eight distribution channels;

FIG. 22 shows the bottom side of the flowrotor of FIG. 21;

FIG. 23 shows the top cross section of the flow rotor of FIG. 21;

FIG. 24 shows the bottom cross section of the flow rotor of FIG. 21;

FIG. 25 is an exemplary flow diagram for implementing one embodiment of the disclosure;

FIG. 26 schematically shows a system according to one embodiment of the disclosure;

FIG. 27 shows an example of a portable instrument according to one embodiment of the disclosure;

FIG. 28 shows the internal view of the system of FIG. 27; and

FIG. 29 shows a cross section of the instrument of FIG. 27.

### DETAILED DESCRIPTION

In on embodiment the disclosure relates to an automated, disposable and simple to use fluidic circuit. The fluidic circuits may define a rotor mountable to a centrifuge and defining a flowrotor. The centrifuge may define a reversible centrifuge. The flowrotor can be configured such that the tangential component of the angular acceleration of the centrifuge can be used to move the fluid to specific sequential reaction chambers while the radial acceleration component is used to keep the fluid in any one of the plural chambers. By timing the rotor action, the fluid can be directed from chamber to chamber. Each chamber may contain one or more reactants which react with the fluid. By controlling the time the fluid resides in each chamber, each chamber reaction can be timed to completion.

Put differently, starting (or reversing) tangential acceleration of the centrifuge, which is caused by angular acceleration, causes the fluid to flow tangentially. In one embodiment, the tangential acceleration starts fluid flow in the circuit. In another embodiment, the fluid flow is initiated only by the centripetal force. The subsequent radial acceleration, caused by the angular velocity, forces the fluid to travel to and then stay in certain (nearest downstream) reaction chambers until the next stop/start cycle, or reversal of rotation direction (negative angular acceleration) causes the fluid to flow out and into the next chamber.

When the centrifuge spins at a given direction at its normal speed (e.g., 3000 rpm which may be the speed required to keep the fluid stable at a given location), a high negative angular acceleration is applied until the rotor spins at a similar speed but in the opposite direction. In one embodiment, an angular acceleration that can fully reverse the flowrotor's rotation direction in about one second is considered as having a high angular acceleration. As the rotor rapidly slows down and approaches zero rotational velocity, the radial (or centripetal) acceleration also approaches zero. The angular, and thus tangential, acceleration is maintained very high throughout this process. When the radial acceleration crosses zero, the tangential acceleration is still very high and it can briefly act on the fluid alone, causing the fluid to move tangentially. If the fluid chamber is appropriately shaped, the fluid can be moved slightly towards the center of rotation and thus cross a barrier that would otherwise maintain the fluid in a stable position under centripetal acceleration only. In an embodiment of the disclosure, advancement of a fluid in a chamber to a next chamber happens when the centripetal acceleration is nearly zero and the angular acceleration is high.

In one embodiment, the fluid path is configured such that the fluid flows just slightly toward the center of rotation in order to exit each reaction location (interchangeably, reaction chamber). In this manner, the fluid can remain stable in each chamber under constant rotational speed unless a reversal of rotation direction rapidly occurs. By having lyophilized or dried reagents immobilized in various reaction chambers along the flow path, reactions can be carried out in a controllable sequence. Further, exposure time for each chamber reaction can be controlled by timing the rotation direction.

In another embodiment, the fluidic circuit may also split and/or combine flows to incorporate more complex protocols. For example, a chemical or biological sample under study can be loaded in a chamber near the rotational center of the circuit. Other required reagents may also be loaded at other locations near the center of the manifold. Using a predetermined sequence of rotations, the centrifuge can automatically execute the assay protocol by virtue of the sequence of programmed rotational events.

Thus, according to one embodiment a small fluidic circuit can be coupled to a centrifuge and utilize a programmable motor and controller to automatically execute a multi-step fluidic assay with no further user intervention after loading the sample carrying fluid. The same fluidic module can also serve as a reaction detection cell, for example, by trapping or immobilizing the final assay product in a controlled location for optical or other detection methods. The centrifuge can be capable of relatively high angular acceleration, such that the inertial forces in the fluid can overcome the viscous forces in the fluid, thus allowing the fluid to flow tangentially relative to the rotating manifold upon rapid acceleration. This movement can only occur when the angular velocity is relatively low, for example, upon start-up, or when reversing direction and crossing a zero speed. The force on the fluid that results from the radial acceleration associated with the angular velocity prevents the fluid from moving towards the center of rotation and escaping a reaction chamber.

In one embodiment, the flowrotor's temperature is regulated using infrared (IR), induction or convection heating. The flowrotor can be equipped with liquid crystal colorimetric temperature indicators with optical detectors for temperature feedback. Alternatively, simple air temperature measurements can be used if convection heating or cooling is applied. Alternatively, IR emission from the flowrotor surface can be used to measure temperature and provide feedback to the temperature regulation system.

Advantageously, the disclosed embodiments enable fluid flow and reaction detection without using pumps, valves, or pressure sources. The lack of complex fluid driving instruments is advantageous in creating a small, low power, portable instrument that can be used without any other complex laboratory equipment. These advantages make the disclosed flowrotor particularly suitable for point-of-care applications.

Mixing of the reactants in each reaction chamber can be implemented by adding a small, oscillating component to the tangential (interchangeably, angular) velocity, while maintaining enough radial acceleration (i.e., angular velocity) to prevent the reactants from leaving their respective chambers. The oscillating component can be applied asymmetrically to ensure that the fluid does not leave the chamber via the normal exit path during mixing.

The flowrotor reaction chambers can be washed by introducing a washing solution after reactions are completed. The centrifuge can be driven such that the spinning or the rapid changing of direction of the flowrotor can advance the cleaning solution. In another implementation parallel processing channels are used to introduce a preloaded washing solution at a designated steps and by joining the wash flow path with the reaction path at designated locations.

A chamber with bi-directional exits (that is, two exists) can be configured to flow the fluid into one exit at a particular step even if the flowrotor is not rotating in the direction required to force the fluid out the desired path.

This can be accomplished by slowly decelerating and then accelerating in reverse direction, as long as the angular (and thus tangential) acceleration is kept low enough to allow the viscous forces in the liquid to dominate the inertial forces and thus not cause the fluid to exit during the reversal of direction. For example, if the direction reversal is slowed down to occur over a 5 to 10 second period, the inertial forces will not be sufficient to move the fluids out of their present chambers.

In another embodiment, an evaluation area is configured in the rotor so as to enable testing. Assay testing can be done by visual inspection (e.g., using known dyes to indicate presence or absence of a reactant) or can require electro-optical components. In an exemplary embodiment, an evaluation area is configured in the flowrotor to visual or instrument inspection. The evaluation area can have a U-shaped channel with a vent/drain hole in the downstream end. The U-shaped channels can be positioned near the perimeter of the disk, in order to allow room for as many of them as possible, and also to allow the option of illuminating the chambers from the edge of the disk.

The vent/drain hole allows air to escape so that the evaluation area can fill completely. The vent hole also allows excess fluid to drain for subsequent flowrotor washing. The excess fluid can be absorbed by an absorbent material that placed radially outward from the vent/drain hole so as to provide a one way fluidic communication with the reaction chamber. This feature also prevents cross-contamination where multiple assays are tested in parallel on the same flowrotor.

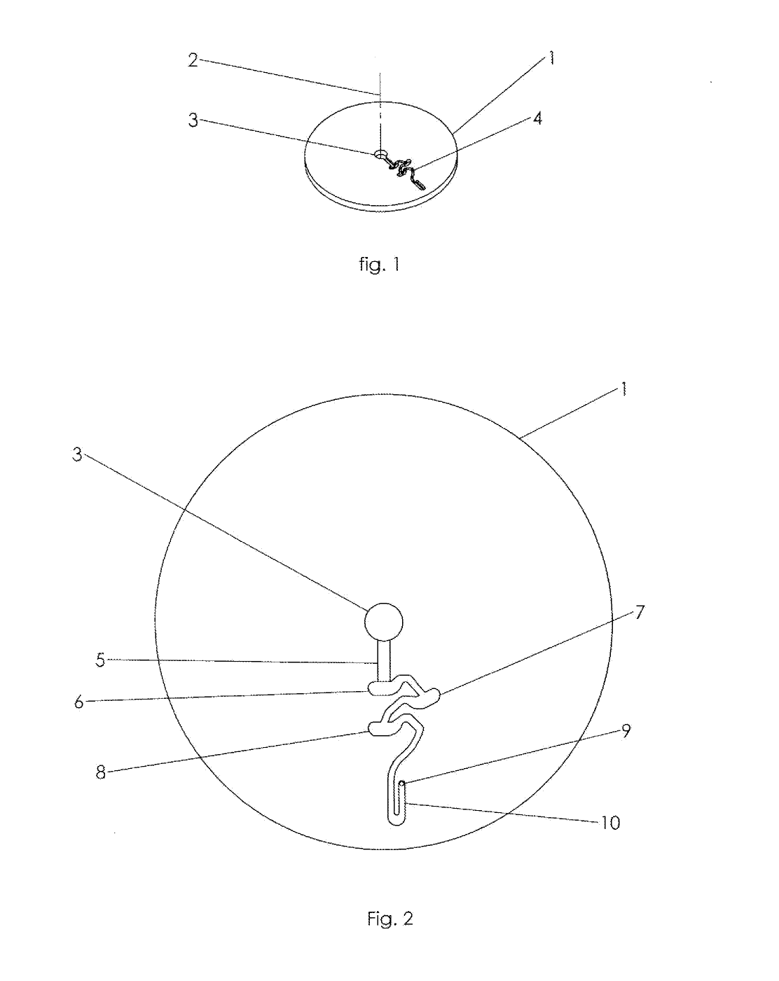

FIG. 1 is a flowrotor having a single assay path according to one embodiment of the disclosure. Specifically, FIG. 1 shows flowrotor with a single assay path 4. As will be discussed in relation to FIG. 2, the flowrotor of FIG. 1 has three chambers and an evaluation chamber. The assay path is shown in all drawings as open on top for clarity, however, the flowrotor would be covered with a thin plastic film and the flow path would be sealed on the top surface. The reagent loading ports near the center may be open for loading reagents. This can be accomplished a number of ways. For example, in one embodiment a thin film is used to heat seal the flowrotor. The reagents can be added to initial chamber 3 of the flowrotor through an opening in the covering film (not shown). The flowrotor rotates around the axis 2.

FIG. 2 is a plan view of the flowrotor part of FIG. 1. Specifically, FIG. 2 is an exploded plan view of the exemplary FIG. 2 is the flowrotor showing the flow path. The exemplary flowrotor 1 has reaction chambers 6,7, and 8. Each chamber has an inlet and an outlet. The reaction chambers are configured to stably retain a fluid when the rotor is under constant rotational velocity. A plurality of conduits connect the reaction chamber. For example, conduit 5 connects initial chamber 3 to reaction chamber 6. The chambers advance the fluid to the next stage when the direction of rotation is quickly reversed in a direction that causes the fluid to move towards the exit conduit. Evaluation chamber 20 is positioned at the radially distal end of flowrotor 1.

Each of chambers 3, 6, 7, 8 and 10 can hold lyophilized or dried reagents. The lyophilized or dried reagents can become active upon contact with the assay fluid. An opening on the top surface film (not shown) over the initial chamber 3 allows introducing the assay to flowrotor 1.

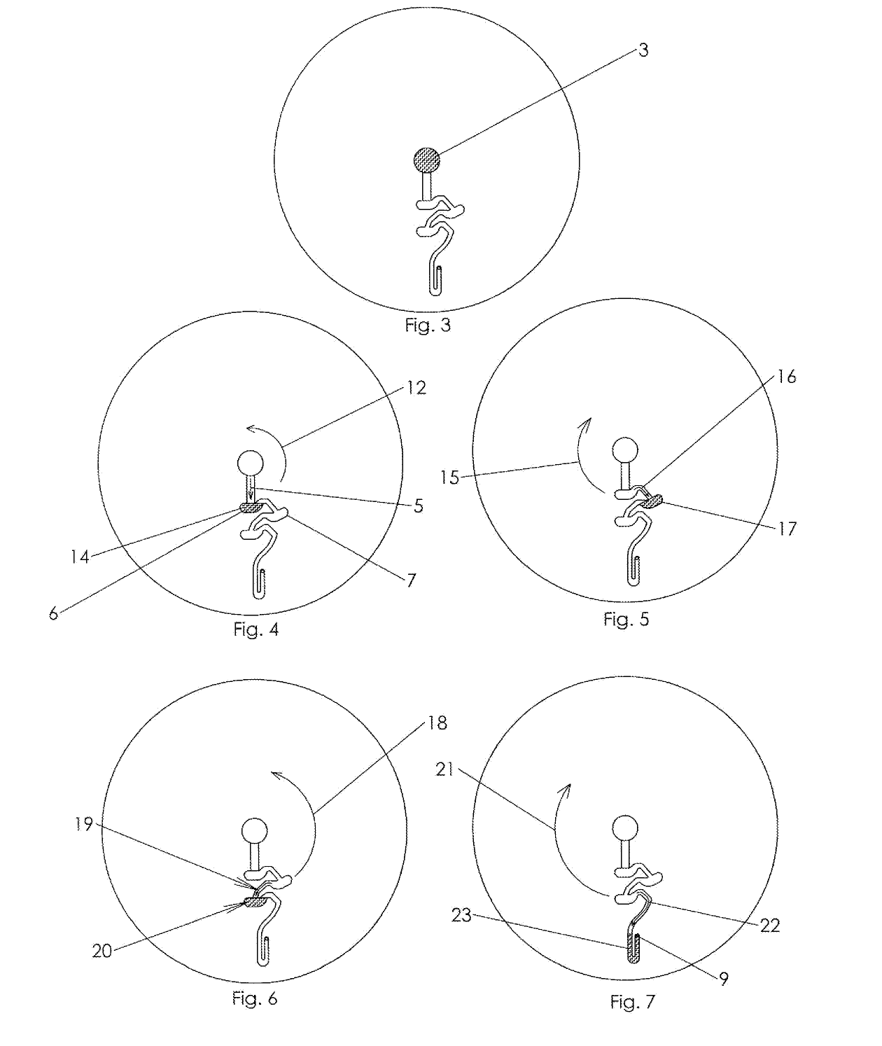

FIG. 3 shows the fluid when introduced into the flowrotor. Specifically, FIG. 3 shows the initial location of the assay fluid (represented by hatching) when first introduced into flowrotor 1. Flowrotor 1 can be stationary at this point, so that the assay fluid can be contained within initial chamber 3. The flow path (or paths) out of initial chamber 3 can be formed above the fill level so that the assay will not leave initial chamber 3 before the flowrotor begins rotating. In one embodiment, the axis of rotation is substantially perpendicular to a surface of the flowrotor.

A vertical axis of rotation is particularly suitable when dried reagents are present in the central chamber and reaction time is needed to dissolve or rehydrated the reagents in the assay. The assay and the reagents may be mixed by introducing small angular oscillations to the rotor. In another embodiment, the combination additionally be heated or cooled to initiate a reaction.

FIG. 4 illustrates the fluid location after the rotor spins for the first time in the counter clockwise (CCW) direction. Specifically, FIG. 4 shows fluid 14 after the rotor spins for the first time in a CCW direction as indicated by arrow 12. As inertial forces act on flowrotor 1, the assay flows radially outward via conduit 5.

In another embodiment where the flowrotor was turning in Clockwise direction, some of assay 14 may flow past reaction chamber 6 and end up in second reaction chamber 7. The fluid may be retained in chamber 6 for a period of time by supplying the same rotational velocity in the same direction. The fluid may also be heated or cooled at chamber 6 or mixed using small variations in angular velocity or direction. Assay 14 may be driven out of chamber 6 by using a second, opposite rotational direction. The second rotational direction may be achieved using high tangential accelerations biased in the CCW direction to ensure that fluid 14 is driven out of chamber 6 completely.

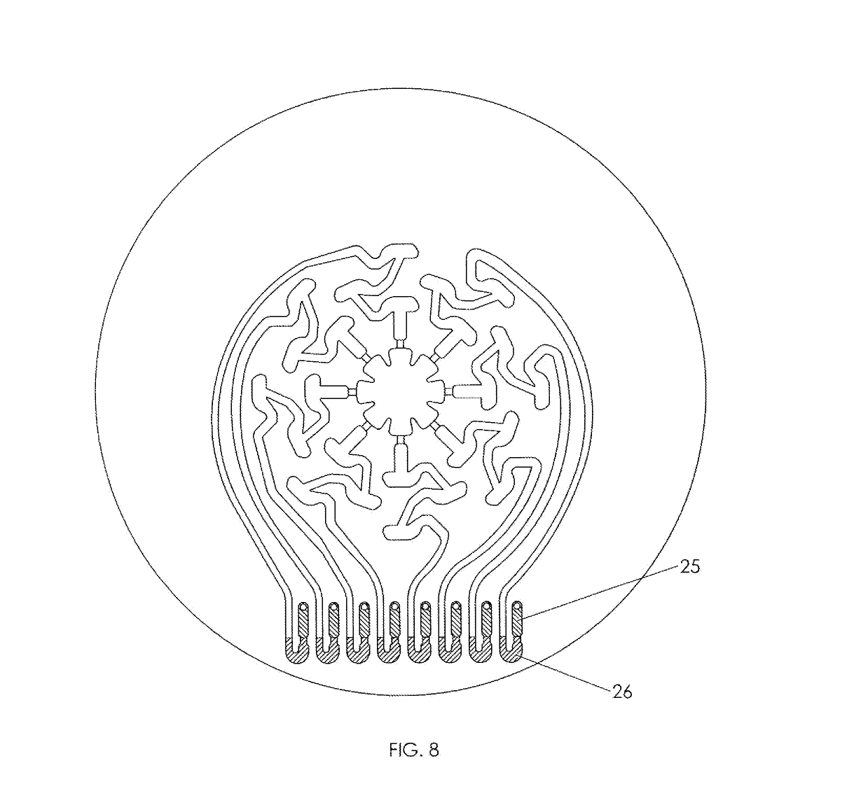

FIG. 5 illustrates fluid relocation by reversing rotation direction. Specifically, FIG. 5 shows the fluid location after the first reversal of rotation direction from CCW to CW. Here, fluid 17 is transported from chamber 14 (FIG. 4) by reversing the rotation direction from CCW 12 to CW 15. In one embodiment, reverse CW acceleration causes the fluid to escape the first chamber 6 and flow into the second chamber 7 through conduit 16 as the rotational velocity passes through zero RPM. The assay fluid may be kept in chamber 17 as long as required by continuing with some rotational velocity in the same CW direction. The fluid may also be heated or cooled or mixed using variations in the flowrotor's angular velocity. A higher rotational velocity can be applied in the CW direction to ensure that the fluid does not escape the chamber prematurely. In an exemplary embodiment, the fluid is maintained in the chamber by applying a rotational velocity of about 1,000 to 4,000 rpm with little or no angular acceleration.

FIG. 6 shows the fluid location after a second reversal of rotation direction from CW to CCW. Specifically, FIG. 6 shows location of the fluid 20 after the second rotation reversal from CW 15 to CCW 18. The reverse CCW acceleration causes the fluid to escape second chamber 7 and flow into third chamber 8 via channel 19 as the rotational velocity passes through zero RPM. The fluid may be kept in this position as long as required by maintaining with some rotation velocity in the same direction CCW. The angular acceleration can be zero. The fluid may also be heated or cooled at this location or mixed using small variations in angular velocity, with higher accelerations biased in the CCW direction to ensure that the fluid does not leave the chamber prematurely.

FIG. 7 shows the fluid location after the third reversal of rotation direction from CCW to CW. Specifically, FIG. 7 shows the location of fluid 23 after a third rotation reversal from CCW 18 to CW 21. The reverse CW acceleration causes the fluid to escape the third chamber 8 and flow into evaluation chamber 10 through connecting conduit 22 as the rotational velocity passes through zero.

Fluid 23 may be maintained at this position as long as required by continuing with some rotational velocity in the same direction CW. The fluid may also be heated or cooled at this location, or mixed using small variations in angular velocity. Excess fluid will flow out from vent 9. The overflow may be captured by a chamber on the far side of the rotor (not shown) or by an absorbent material (not shown) positioned at the radially distal end of the flowrotor.

FIG. 8 shows a multi-channel flowrotor according to one embodiment of the disclosure. Specifically, FIG. 8 illustrate a flowrotor design with a central sample input area 27, which feeds eight separate flow paths so that eight separate reactions or assays may be run on a single sample. In one embodiment, the fluid channels are configured such that the assay evaluation areas 25 can be close to each other so that a single imaging instrument can view all eight results simultaneously.

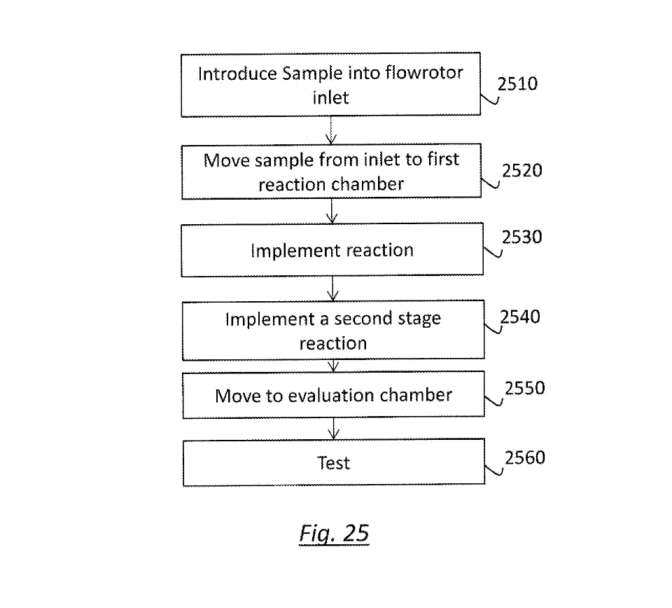

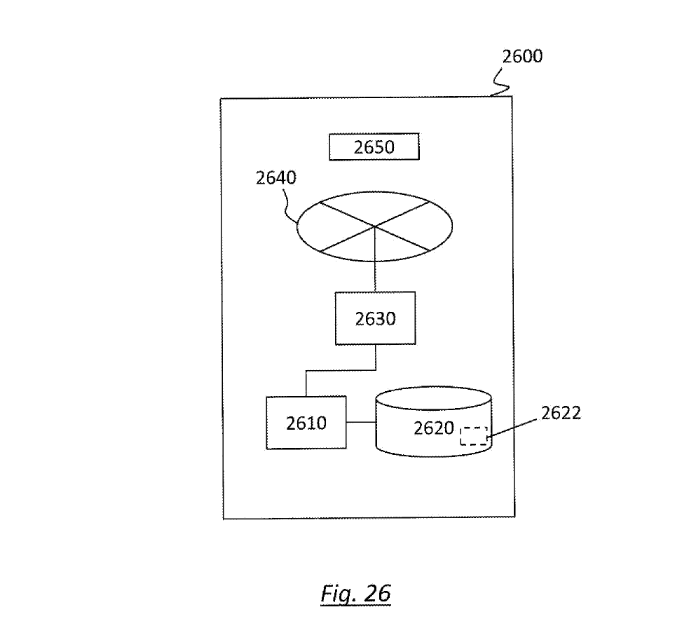

The multi-channel flowrotor of FIG. 25 may be used for a DNA assay using protein capture. For example, the flowrotor can be used automate a digital DNA test. A purified DNA sample, extracted from blood or tissue or recovered from other material can be introduced to initial chamber 27 of the flowrotor. When the process is started, the rotor rotates CCW and the sample is evenly distributed to the first downstream reaction chambers 116 via conduit 92. The first chambers can contain a dried mixture of reagents necessary to amplify a relevant segment of the DNA under study.

When dried reagents are dissolved (partially or completely) the flowrotor reverses direction via application of a negative angular acceleration and the fluids move to a second chamber 117. The second chamber may contain primers to allow the DNA amplification. DNA amplification occurs when a segment of DNA that matches the primers is present in the sample. When amplification process is complete, the flowrotor reverses rotation direction, and the fluid moves to a third chamber. The third chamber may contain a dried dye to stain any double stranded DNA that may have been amplified. In this manner, the dye acts as an indicator. When the dye has dissolved or reacted, the rotor may rapidly reverse rotation direction again to move the fluid into evaluation area 25.

In one embodiment, evaluation area 25 is preceded by a protein capture (interchangeably, sequester) area 25 where proteins left over from the amplification reaction are selectively sequestered. Since the proteins may also absorb the dye, protein capture area 26 can serve as a positive control for each flow channel. That is, each channel can show the dye color in protein capture area 26 or it can be assumed that the reaction or fluid flow did not occur and result is null. Downstream of the protein capture area is evaluation area 25. Assay evaluation area 25 may contain one or more pretreated surfaces to absorb any double-stranded, dyed DNA.

Through inspection at evaluation area 25 any color change in the assay can be detected. Inspection can be done visually or with the aid of an instrument. An exemplary instrument is a smartphone containing appropriate image processing application(s). Dye visible in evaluation area 25 downstream of protein sequester area 26 can indicate a positive test for the DNA segment that matches the primer present in that channel.

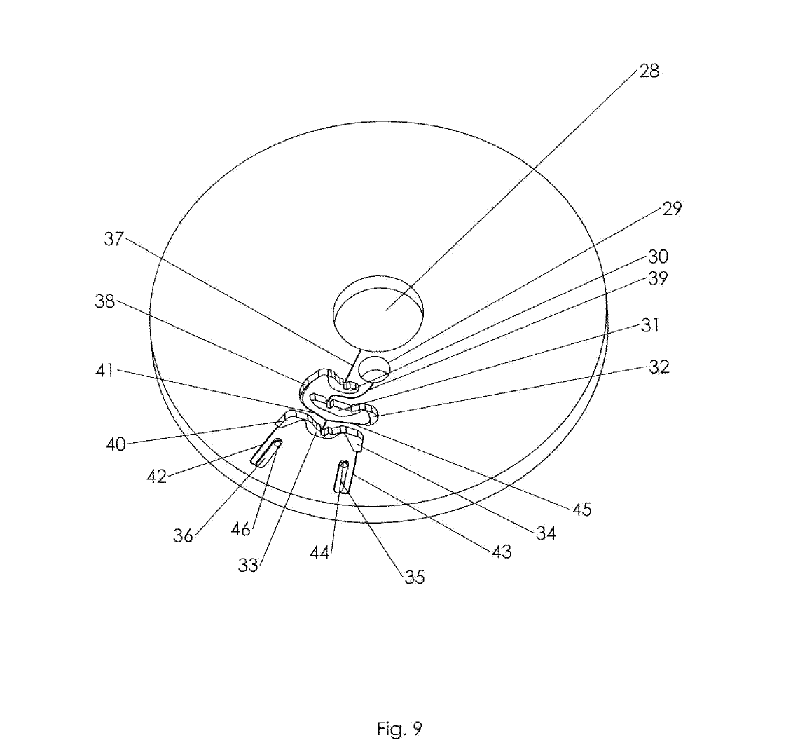

FIG. 9 illustrates an exemplary embodiment having bifurcated flow path. Specifically, FIG. 9 shows a plan view of a flowrotor with two separate reagent flow paths originating from central starting areas 28 and 29 that eventually converge in chamber 33. Central area 28 is where a sample can be loaded. The sample can be driven through conduit 37 to chamber 30 upon spinning the flowrotor radially in either direction. Another reagent or another sample can be loaded into area 29 which would be driven through conduit 39 to chamber 31. Fluid movement occurs upon radial spinning. Any fluid in chamber 31 can be driven out via conduit 38 to chamber 44 by adequate counterclockwise CCW acceleration. Fluid present in chamber 31 can be partially driven out conduit 45 to chamber 44 by adequate clockwise CW acceleration. The amount of fluid driven out is limited in volume by the metering area 32.

Fluid present in chamber 44 can be driven either by an adequate CC acceleration into chamber area 34 or by an adequate CCW acceleration into chamber 40. As long as rotation continues in either direction any fluid in chambers 34 and 40 will drain via conduits 43 and 42 to evaluation chambers 35 and 36, respectively.

The excess fluid may drain from evaluation chambers via drain holes 44 and 46. There is a relationship between the direction of the angular acceleration and where the fluid goes: the inertia of the fluid moves the fluid in a direction opposite direction of the acceleration. That is, inertia always opposes acceleration. Using this relationship, the flowrotor can be accelerated in different directions to control fluid movement into the appropriate chamber.

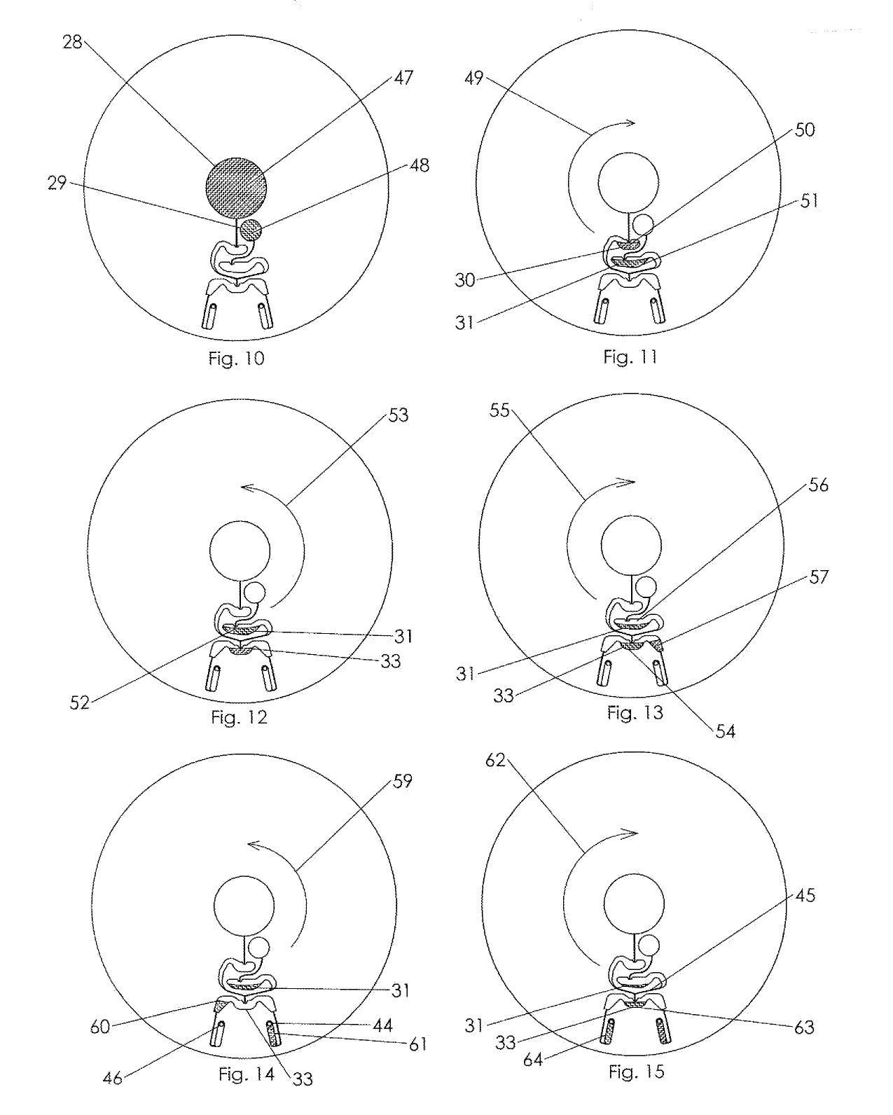

FIGS. 10-20 show various fluid movement phases through the flowrotor according of FIG. 9. Specifically, FIG. 10 shows the flowrotor of FIG. 9 with a first fluid 47 loaded into starting area 28, and a second fluid 46 loaded into starting area 29. Loading fluids 47 and 48 into the flowrotor can be done with any conventional fluid delivery means. Each of fluids 47 and 48 can define a DNA sample, a reagent, a polymerase solution or any other fluidic carrier.

In one embodiment, the fluid is loaded onto the flowrotor prior to assembling the flowrotor on the centrifuge. In another embodiment, the flowrotor is loaded with fluid after the flowrotor is assembled on the centrifuge. The centrifuge can be a conventional centrifuge. In another embodiment, the centrifuge can comprise a motor, a housing and a controller. The controller can be a programmable controller having a microprocessor circuitry in communication with a memory circuitry. The controller can be programmed to rotate the flowrotor at predefined speeds, acceleration and duration.

FIG. 11 shows the flowrotor of FIG. 10 after the first spin has started. The centrifugal acceleration causes the first fluid to move from starting area 28 to chamber 30 and is shown as first fluid 50. FIG. 11 also shows the new second fluid location 51 moved from starting area 29 to chamber 31. The spin direction 49 is CC, although either spin direction can result in the same fluid transfer.

FIG. 12 shows the repositioning of the fluids in the flowrotor of FIG. 11 after spin direction 53 has been quickly reversed from CC to CCW 53. First fluid 54 has moved from chamber 30 to chamber 33 due to the inertial forces exerted by the fluid as rotation direction is rapidly reversed to CCW. The second fluid does not move because a viable exit path from chamber 31 does not exist when the flowrotor is accelerating CCW.

FIG. 13 shows positions of the different fluids of FIG. 12 after spin direction 55 has been quickly reversed from CCW back to CW. The inertial forces of the first fluid have moved it from chamber 33 to side-chamber 34, the fluid shown as 57. Continued spinning drains the first fluid into evaluation chamber 35. At the same time, the second fluid 56 and 58 has partially transferred from chamber 31 to chamber 32. The second fluid then drains into chamber 33 after the first fluid is pushed out. The second fluid's draining can be delayed by routing the second fluid through conduit 45.

FIG. 14 shows the position of the fluids in the flowrotor of FIG. 13 after the spin direction 59 has been quickly reversed from CW back to CCW. The inertial forces of the second fluid located in chamber 33 cause it to move from chamber 33 to side-chamber 40. Here, the second fluid shown with the hatching 60. Continued spinning drains the second fluid 60 into evaluation chamber 36. Any excess second fluid exits through drain hole 46. The second fluid remaining in chamber 31 does not move to a new location because there is no viable exit from chamber 31 when the flowrotor is accelerating CCW.

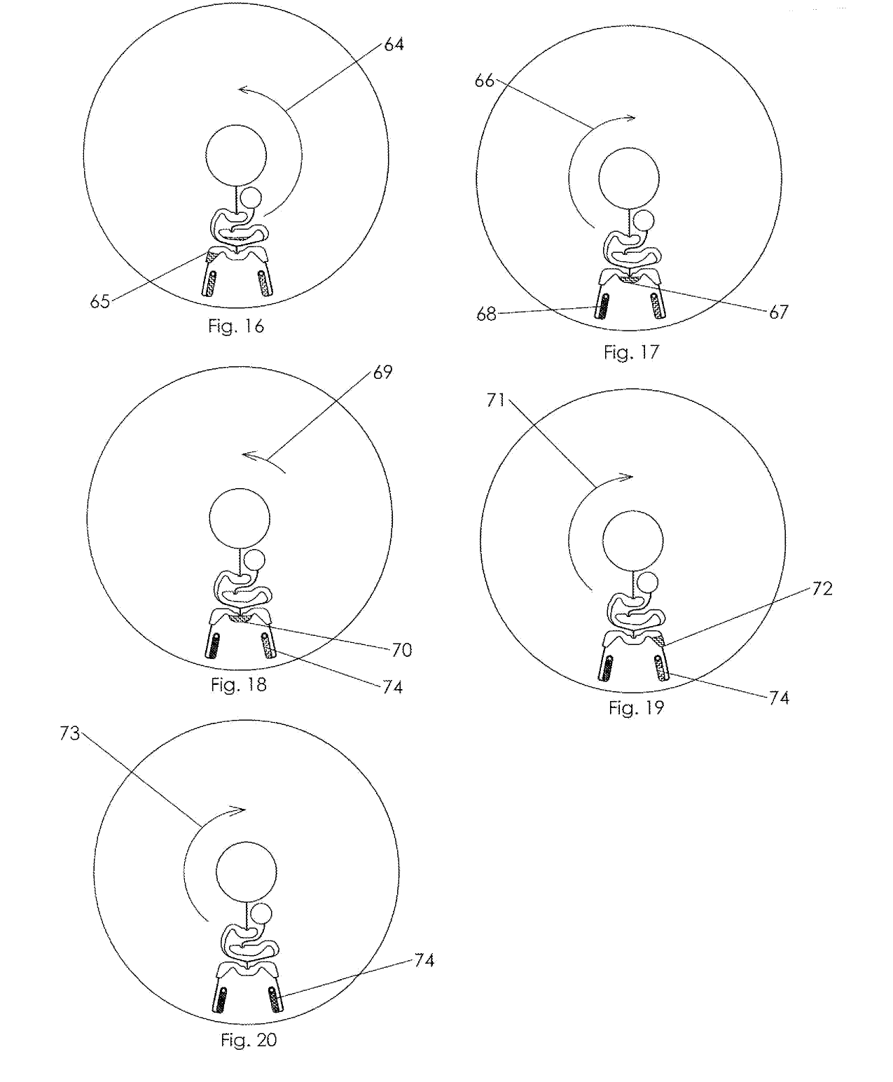

FIG. 15 shows the position of the fluids in the flowrotor of FIG. 14 after the spin direction 62 has been quickly reversed from CCW back to CW. The second fluid 63 has partially transferred from chamber 31 to 32, and then drained into chamber 33, via conduit 45.

FIG. 16 shows the position of the fluids in the flowrotor of FIG. 15 after the spin direction 64 has been quickly reversed from CW back to CCW. Here, the inertial forces of the second fluid located in chamber 33 cause it to move from chamber 33 to side-chamber 40. In FIG. 16, the second fluid is shown with hatching 65. Continued spinning drains the second fluid into evaluation chamber 36 with any excess fluid exiting through drain hole 46. Any second fluid that remaining in chamber 31 does not move to a new location case because there is no viable exit path from chamber 31 when the flowrotor is accelerating CCW.

FIG. 17 shows the position of the fluids in the flowrotor of FIG. 16 after the spin direction (65) has been quickly reversed from CCW back to CW. The remaining second fluid (67) has transferred from chamber (31) to (32), and then drained into chamber (33), via conduit (45).

FIG. 18 shows the position of the fluids in the flowrotor of FIG. 17 after the spin direction 69 has been gently reversed from CW back to CCW. Because of the gentle acceleration, the inertial forces of the second fluid 70 located in chamber 33 are small compared to the viscous forces the fluid does not move into another chamber.

FIG. 19 shows the position of the fluids in the flowrotor of FIG. 18 after the spin direction 71 has been quickly reversed from CCW back to CW. The remaining second fluid 72 is transferred from chamber 33 to 34, and then drained into chamber 35 through conduit 43. Excess fluid exits through drain hole 44.

FIG. 20 shows the position of the fluids in the flowrotor of FIG. 19 after the CW spin has continued for an additional duration. The fluid that was in chamber 34 has now drained into evaluation chamber 35 via conduit 43 which is shown with hatching 74. The fluid displaces the fluid that was already in that location by pushing it out through drain hole 44.

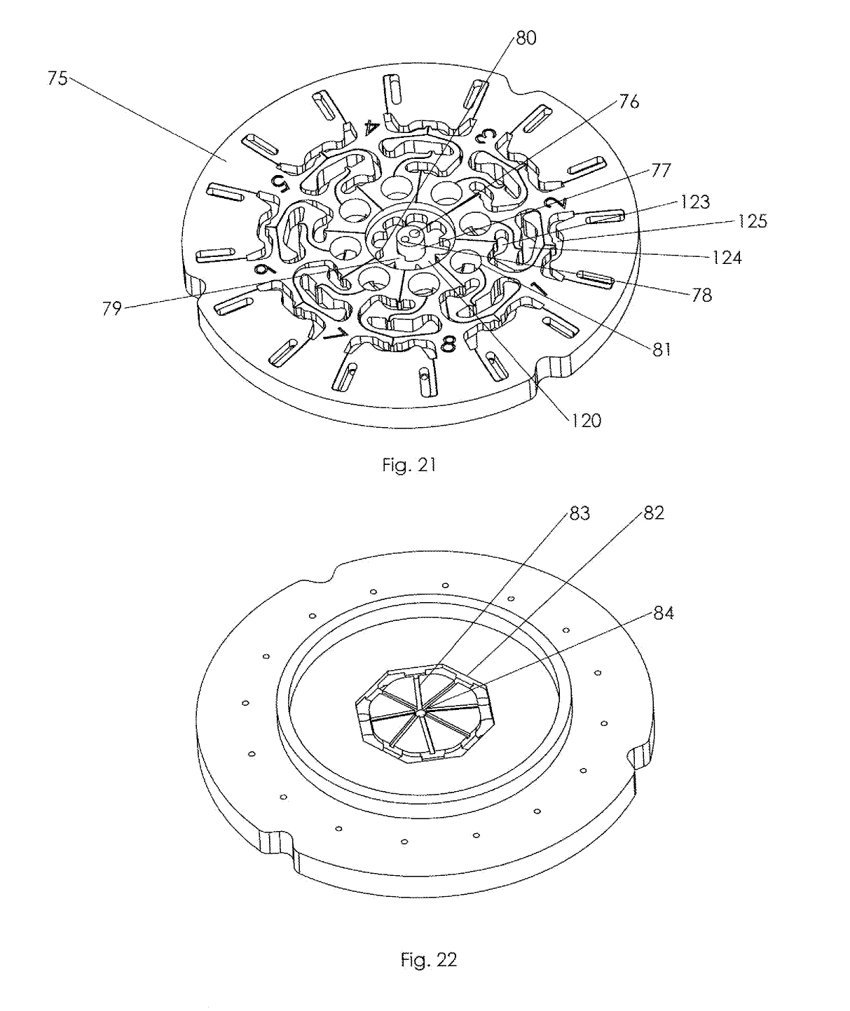

FIG. 21 shows an exemplary flowrotor having two filling ports and eight distribution channels. Specifically, FIG. 21 shows flowrotor 75 having 8 instances of the fluidic channel design where each channel is similar to the flowrotor of FIG. 9. The instances are marked on FIG. 21 with numbers 1-8. Flowrotor 75 has a central distribution chamber 120 surrounded by a rosette-shaped port 80 configured to evenly distribute the fluid across the central region to ensure even filling of all the channels. While a rosette-shaped port is used, the disclosure is not limited thereto and any other port configured for even distribution can be used.

Flowrotor 75 has filling tower 77 in contact with the sealing film layer (not shown). The film can have holes it to allow access to two filling ports 76 and 78. Channel 79 allows the base of port 80 to be in fluidic communication with the reagent filling port 78. Flowrotor 75 includes a second distribution chamber for a second reagent. The second distribution chamber, can be positioned on an opposite surface of the (e.g., bottom) of the flowrotor and can be sealed with a second sealing film. The second distribution chamber can be in fluidic communication with filling port 76. The thin films may be heat- or pressure-sealed to top and bottom surfaces of flowrotor 75.

FIG. 22 shows the under surface of the flowrotor of FIG. 21. Here, the bottom end 84 of filling port 76 can be in fluidic communication with a star pattern of channels 83 to distribute fluid to ring channel 82. Ring channel 82 can be in fluidic communication to the second reagent initial fluid reservoirs 81.

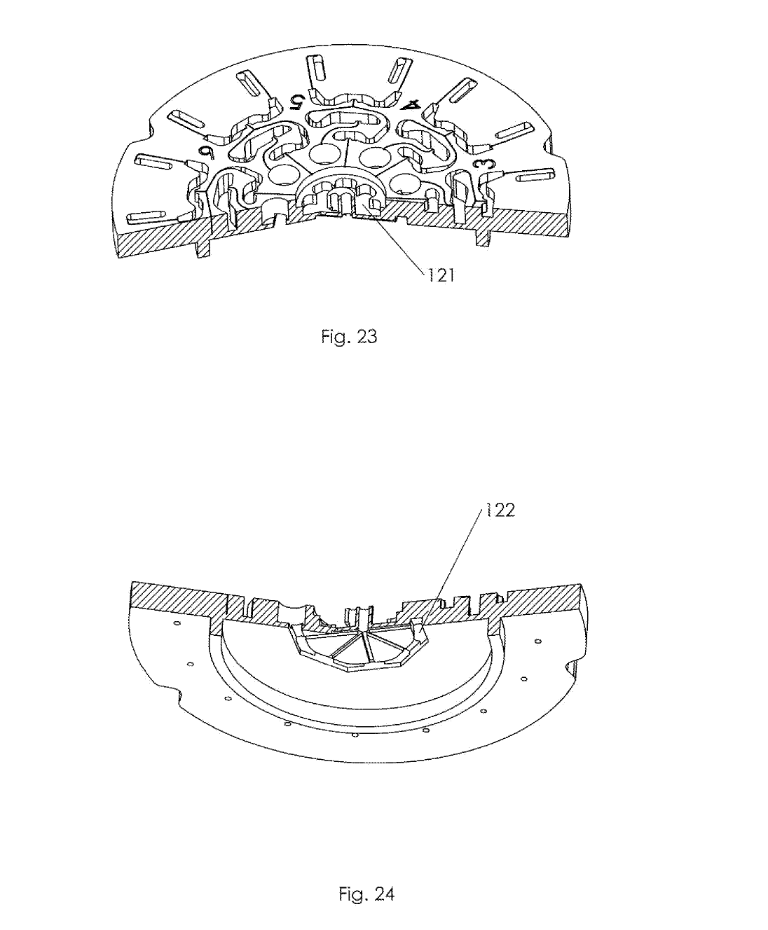

FIG. 23 shows a cross section of the flow rotor of FIG. 21. FIG. 23 illustrates the geometry of the two central distribution systems allowing two central fill points to equally distribute two separate fluids to a plurality of assay paths. In FIGS. 21 and 23, there 8 distribution channels are shown although more or less channels can be used without departing from the disclosed principles.

FIG. 24 shows an inverted cross section of the flowrotor of FIG. 21. Specifically, FIG. 24 shows the design details of the two central distribution systems, one on top 121 and one on the bottom 122.

The following examples show an application of the disclosed embodiments to an assay using SiO₂ DNA capture. Accordingly, reference is made to the embodiment of FIG. 9, where the flowrotor was used to automate a digital DNA test.

In the exemplary application, a purified DNA sample was extracted from blood or tissue or recovered from other material and was introduced to initial chamber 30 of the flowrotor in a buffered solution. Chamber 30 contained a lyophilized isothermal DNA amplification reagent. The amplification agent is rehydrated when the buffer/sample mixture is introduced. The second reagent position 122 is filled with a wash buffer.

When the process is started, the rotor is gently oscillated in order to fully reconstitute and mix the lyophilized reagent with the sample. In one embodiment, rotor is oscillated at a frequency of about 5 Hz at an angular acceleration of about 10 to 50 radians per second squared. Once the first reagent was fully mixed, the flowrotor was accelerated in CW direction. The acceleration causes the sample (the first fluid) and the wash buffer (the second fluid) to be evenly distributed to first downstream chambers 34. In one implementation, the sample fluid goes into first reaction chambers 123, and the wash fluid goes into chambers 84. Each first reaction chamber 123 contains a dried DNA primer and required reagent salts so as to only amplify the sample DNA if a particular, matching sequence was present in the sample.

Further, each of first reaction chambers 123 contained different oligo primer sets. The different primers allow each fluid path to perform a different DNA test. Thus, simultaneous with transferring the first fluid to the first chamber, the wash fluid can be evenly distributed into the wash reservoirs 81.

Once the primers and the reagent salts were dissolved, amplification was given time to complete. The samples were then transferred to the SiO₂ capture chambers (alternatively, convergence point 33) by quickly reversing the rotation to CCW. The SiO₂ capture chambers contained immobilized, hydrolyzed SiO₂, and a dried reagent salt known to cause DNA molecules to attach. The reagents were given sufficient bind time while the flowrotor continued to spin. This ensures that all the reagents stayed in place while binding was occurring. Small velocity oscillations were added to the one-way spinning to encourage mixing of the reaction. Oscillations with angular accelerations in the range of 10-50 radians per second square (RPS) are suitable for such mixing.

Once the binding reaction was complete, rotation direction was quickly reversed to CW to move the first reagent solution into evaluation chambers 35 and 36. The solution contained all the materials that did not bind to the SiO₂, including unused proteins that were required for amplification. Evaluation chambers 35, 36 contained a non-specific binding reagent, such as hexadecyl trimethyl ammonium bromide (CTAB), and a dye, such as Nile Blue. Any protein present in the solution attached to the surface and to the dye. While the SiO₂ chamber was draining it was also being refilled with the wash reagent which diluted the salt and caused the bound DNA to go into the solution.

Once the bound DNA in the SiO₂ capture chamber is released into the solution and also the solution in first evaluation chamber 35 dissolved the dye and bind to the surface the rotation was quickly reversed to CCW. The solution is thus moved into the second evaluation chamber 36. The second evaluation chamber also contained a non-specific binding reagent such as CTAB and a dye. If there was DNA in the solution (indicating a positive test result) the DNA and the dye would bind to the surface. Once the solution in second evaluation chamber 36 had enough time to dissolve the dye and bind, the rotation is quickly reversed to CW causing the wash reservoir 124 to release more wash reagent into the SiO₂ chamber.

The flowrotor rotation is quickly reversed again to CCW, causing the wash in the SiO₂ chamber to transfer to the second evaluation chamber pushing out any non-immobilized material and leaving behind bound, dyed DNA. An exemplary rapid rotation can be in the range of 2,000-4,000 RPM. Another exemplary rapid rotation can be about 3000 RPM. The rotation reversal created a positive result for the DNA test only if any new dye was present. Otherwise the dye will can be washed out.

The flowrotor rotation was then quickly reversed to again CW rotation to transfer any remaining wash in the reservoir to transfer to the SiO₂ chamber.

The rotation direction of the flowrotor was then gently reversed to CCW so that the wash in the SiO₂ chamber remained in the chamber. This is so that wash solution will again go into the evaluation chamber 74 rather than chamber 68 upon a subsequent rapid reversal of rotation direction.

The flowrotor was then quickly reversed direction to CW so that the wash in the SiO₂ chamber would move to the first evaluation chamber to wash out any unbound material from that chamber. The dye will only remain if double stranded DNA was present in the wash.

FIG. 25 is an exemplary flow diagram for implementing one embodiment of the disclosure. The flow diagram of FIG. 25 can be implemented using a flowrotor in conjunction with a housing for receiving the flowrotor. The housing can have a motor to rotate the flowrotor about an axis. The housing can also have a programmable controller (e.g., microprocessor in communication with a memory circuit) to control the rotation direction, rotation acceleration, speed and duration of the motor rotation.

At step 2510, a sample fluid is introduced into the initial chamber of a flowrotor. The sample fluid can be, for example, a sample containing one or more DNA segments. The sample fluid can be introduced through the housing, for example, by having a cartridge or a test tube (not shown) configured to deliver the sample to the flowrotor. The sample may also be manually delivered to the flowrotor.

At step 2520 the sample is moved from the initial chamber to the first reaction chamber. The fluid movement can be caused by the motor rotating the flowrotor about an axis. The fluid inertia can cause the sample to move from the initial chamber through a conduit connecting the initial chamber with the first reaction chamber. In one embodiment, the flowrotor is rotated in a first direction to provide a first radial acceleration to move the sample fluid from the initial chamber to a first reaction chamber. The first reaction chamber can have an inlet and an outlet distal from each other. The first reaction chamber may also have one or more reactants deposited therein. The reactants can be in solid form.

Once the sample fluid reaches the first reaction chamber, at step 2530, the sample fluid is retained in the first reaction chamber to implement one or more reactions. The sample may be retained by continuing or maintaining a rotational speed (angular velocity) sufficient to keep the sample in the first reaction chamber. The sample fluid can be held at the first chamber by programming the motor to maintain angular velocity for a sufficient duration. In an exemplary embodiment, the first chamber may have a lip designed to retain the fluid. The first reaction chamber be also be heated or cooled while the sample fluid is held. Additional mechanical agitation may be provided by changing the rotation direction at a speed that does not move the sample fluid.

At step 2540 the reacted sample can be moved to a second reaction chamber to allow subsequent reactions. For example, the first stage reaction may comprise amplification of a DNA strand while the second stage reaction may comprise fluorescent tagging of the amplified DNA. The second stage may be optional. Additional stages can be added consistent with the disclosed embodiments. The movement of the reacted sample from one chamber to another chamber can be implemented by rapidly accelerating the flowrotor at a negative angular acceleration relative to the immediately preceding angular velocity until the angular velocity is reversed.

At step 2550 the reacted sample is delivered to the evaluation chamber and at step 2560 a test is conducted to determine the results. The evaluation chamber of the flowrotor can be configured to communicate with a test instrument. For example, the evaluation chamber can be configured to enable one or more optical instruments to detect a fluorescence dye in the reacted sample. Additional, unloading, washing or loading steps may be implemented as disclosed above.

FIG. 26 schematically shows a system according to one embodiment of the disclosure. Specifically, FIG. 26 shows housing 2600 having flowrotor 2640, motor 2630, controller, 2610 database (memory circuit) 2620 and test instrument 2650. For simplicity, the fluidic circuit is not shown in FIG. 26. Controller 2610 can comprise one or more processor circuits in communication with the database 2620. Database 2620 may include one or more instruction files 2622. The instruction files direct controller 2610 with various rotation and timing instructions. For example, controller 2610 can direct motor 2630 to start rotating flowrotor 2640 in a CW direction. Controller 2610 may also instruct the motor to ramp up/down the rotational speed according to a pre-programmed timing. The acceleration rate can be set through an external interface (not shown) where the operator (or another computing device) determines radial acceleration, radial declaration, angular velocity, direction and duration of rotation.

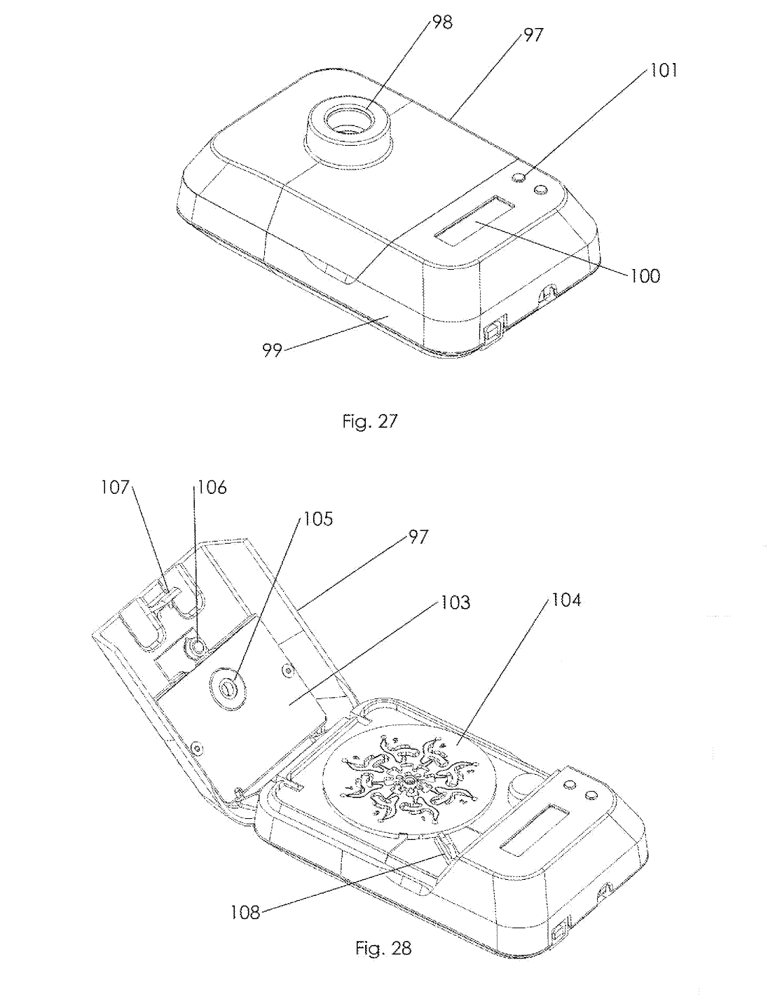

FIG. 27 shows an example of a portable instrument according to one embodiment of the disclosure. Specifically, FIG. 27 shows an example of a portable instrument designed to process a flowrotor assay. The test fluid is delivered via a sample tube that fits into opening 98 in cover 97. Cover 97 is centrally located to the flowrotor. A display 100 and buttons 101 are available as an optional user interface. Other means for interfacing the instrument may include a conventional port, such as a USB port. That chassis 99 contains the necessary electronics and mechanical parts for processing the assay.

FIG. 28 shows the internal view of the system of FIG. 27. Specifically, FIG. 28 shows the instrument of FIG. 27 with the cover 97 open to show access to the flowrotor area. A latch 107 holds the cover closed during operation. Heater 105 (for heating the sample) can be centrally located on the cover. A convection heater plate 103 can be positioned on the cover so that it is close to the surface of flowrotor 104 when the cover is closed. An infrared (IR) sensor 106 monitors the temperature of the flowrotor and provides feedback for controlling heat delivery to heater plate 103. A light pipe 108 illuminates the perimeter of the flowrotor so that the fluorescent dye in the assay can be detected by an optical sensor mounted below the edge of the flowrotor (not shown).

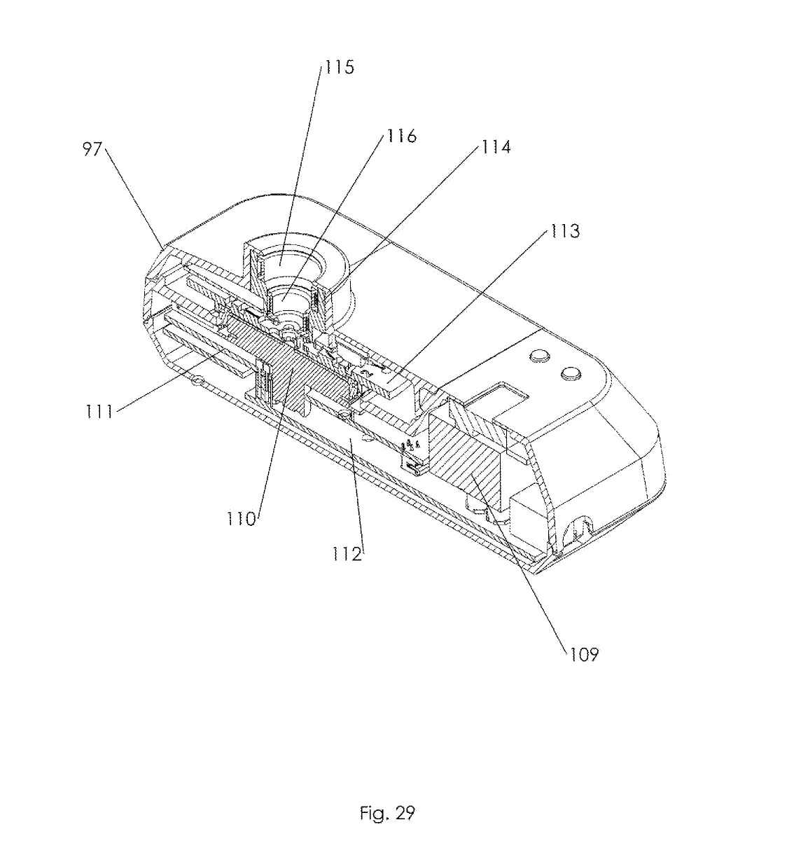

FIG. 29 shows a cross section of the instrument of FIG. 27. Here, two heating elements 115 and 116 that provide heat to the sample tube area. Motor 110 is mounted on a PC board 111 to provide rotational motion to the flowrotor 113. PC board 112 contains a microcontroller (not shown) that controls the instrument. A battery 109 provides the electrical energy to power the instrument.

The following examples pertain to further embodiments of the disclosure. Example 1 includes a method for processing an assay with a radially configured flowrotor, the method comprising: introducing a sample fluid to an inlet of the flowrotor; rotating the flowrotor in a first direction to provide a first radial acceleration to move the sample fluid from the inlet to a first reaction chamber, the first reaction chamber having an inlet and an outlet and containing a first reactant; retaining the assay at the first chamber for a first duration by maintaining a first angular velocity to induce a reaction with the first reactant and to provide a first reacted assay; accelerating rotation of the flowrotor at a negative angular acceleration relative to the first angular velocity until the first angular velocity is reversed thereby moving the first reacted assay from the first chamber to a second reaction chamber, the second reaction chamber having an inlet and an outlet and containing a second reactant; retaining the first reacted assay at the second chamber for a second duration to react with the second reagent to form a second reacted assay.

Example 2 includes the method of example 1, wherein accelerating rotation of a flowrotor at a negative angular acceleration further comprises rapidly accelerating rotation of the flowrotor.

Example 3 includes the method of example 1, further comprising retaining the assay at the first chamber by terminating the first acceleration while maintaining a first rotational velocity of the flowrotor.

Example 4 includes the method of example 1, wherein retaining the assay at the first chamber for a first duration further comprises insitu heating or cooling at the first chamber.

Example 5 includes the method of example 1, wherein retaining the assay at the first chamber for a first duration further comprises insitu agitation of the assay by continually reversing the angular acceleration direction.

Example 6 includes the method of example 1, wherein the first reagent defines a mixture for amplifying a segment of a DNA in the assay.

Example 7 includes the method of example 1, wherein the second reagent is a dye.

Example 8 includes the method of example 1, further comprising optically evaluating the final assay.

Example 9 includes a flowrotor for assay detection, comprising: a disc having an enclosure formed by an upper surface and a lower surface, the enclosure defining a first channel having an inlet, a reaction chamber and an evaluation chamber connected by a plurality of conduits; the disc configured to (1) rotate about an axis at a first direction with a first radial acceleration to move an assay from the inlet through a first conduit to a first reaction chamber, (2) retain the assay at the first reaction chamber by maintaining a first angular velocity to induce a reaction with a first reactant and to provide a first reacted assay, (3) accelerate rotation at a negative angular acceleration relative to the first angular velocity until the first angular velocity is substantially reversed thereby moving the first reacted assay from the first reaction chamber to an evaluation chamber.

Example 10 includes the flowrotor of example 9, wherein the first reaction chamber further comprises a lip to retain the assay therein.

Example 11 includes the flowrotor of example 9, wherein the disc is further configured to retain the assay at the first reaction chamber by terminating the first acceleration while maintaining a first rotational velocity for the flowrotor.

Example 12 includes the flowrotor of example 9, wherein the disc is further configured to heat or cool the assay.

Example 13 includes the flowrotor of example 9, wherein the disc is further configured to provide insitu agitation of the assay by continually reversing rotation.

Example 14 includes the flowrotor of example 9, further comprising a second channel having a second reaction chamber and a second evaluation chamber.

Example 15 includes the flowrotor of example 14, wherein the second reaction chamber fluidically communicates with the first reaction chamber.

Example 16 includes the flowrotor of example 14, wherein each of the first reaction chamber and the second chamber contains a different reagent.

Example 17 includes the flowrotor of example 9, wherein the first reaction chamber comprises at least one of an immobilized lyophilized or dried reagents.

Example 18 includes a detection system, comprising: a motor; a flowrotor; and a controller in communication with the motor, the controller configured to provide the motor with instructions comprising: rotate the flowrotor in a first direction with a first radial acceleration to move an assay from the inlet to a first reaction chamber of the flowrotor; retain the assay at the first reaction chamber by maintaining a first angular velocity to induce a reaction with a first reactant to thereby provide a reacted assay; accelerate the flowrotor's rotation at a negative angular acceleration relative to the first angular velocity until the first angular velocity is substantially reversed thereby moving the reacted assay from the first reaction chamber to an evaluation chamber.

Example 19 includes the system of example 18, wherein the first reaction chamber further comprises an inlet and an outlet and wherein the first radial acceleration delivers the assay to the inlet of the reaction chamber and the negative angular acceleration removes the reacted assay from the outlet of the reaction chamber.

Example 20 includes the system of example 18, wherein the first reaction chamber further comprises a lip to retain the assay.

Example 21 includes the system of example 18, wherein the flowrotor retains the assay at the first reaction chamber by terminating the first angular acceleration while maintaining a first rotational velocity for the flowrotor.

Example 22 includes the system of example 18, wherein the flowrotor is further configured to heat or cool the assay.

Example 23 includes the system of example 18, wherein the flowrotor is further configured to provide insitu agitation of the assay by continually reversing angular acceleration direction.

Example 24 includes the system of example 18, wherein the flowrotor further comprises a second channel having a second reaction chamber and a second evaluation chamber.

Example 25 includes the system of example 24, wherein the second reaction chamber fluidically communicates with the inlet.

Example 26 includes the system of example 24, wherein each of the first reaction chamber and the second chamber contains a different reagent.

Example 27 includes a computer-readable storage device containing a set of instructions to cause a motor to perform a process comprising: rotate a flowrotor in a first direction with a first radial acceleration to move an assay from the inlet to a first reaction chamber, the first reaction chamber having an inlet and an outlet and containing a first reactant; retain the assay at the first reaction chamber for a first duration by maintaining a first angular velocity to induce a reaction with the first reactant and provide a reacted assay; accelerate the flowrotor at a negative angular acceleration relative to the first angular velocity until the angular velocity is reversed thereby moving the reacted assay from the first reaction chamber to a second chamber; retain the reacted assay at the second chamber for a second duration to induce a reaction with a second reagent to form a final assay; and rotate the flowrotor at a second radial acceleration to move the final assay to an evaluation chamber.

Example 28 includes the computer-readable storage device of example 27, wherein the instructions further cause the motor to perform rapidly accelerate the flowrotor at a negative angular acceleration.

Example 29 includes the computer-readable storage device of example 27, wherein the instructions further cause the motor to retain the assay at the first chamber by terminating the first acceleration while maintaining the first rotational velocity of the flowrotor.

Example 30 includes the computer-readable storage device of example 27, wherein the instructions further cause a heating or a cooling device to provide insitu heating or cooling to the first chamber.

Example 31 includes the computer-readable storage device of example 27, wherein the instructions further cause the motor to agitate the assay by continually reversing the angular acceleration direction.

Example 32 includes the computer-readable storage device of example 27, wherein the instructions further cause an optical system to evaluate the final assay.

While the principles of the disclosure have been illustrated in relation to the exemplary embodiments shown herein, the principles of the disclosure are not limited thereto and include any modification, variation or permutation thereof.

What is claimed is:

1. A method for processing an assay with a radially configured flowrotor, the method comprising:
introducing a sample fluid to an inlet of the flowrotor;
rotating the flowrotor in a first direction to provide a first radial acceleration to move the sample fluid from the inlet to a first reaction chamber, the first reaction chamber having an inlet and an outlet and containing a first reactant;
retaining the assay at the first chamber for a first duration by maintaining a first angular velocity to induce a reaction with the first reactant and to provide a first reacted assay;
accelerating rotation of the flowrotor at a negative angular acceleration relative to the first angular velocity until the first angular velocity is reversed thereby moving the first reacted assay from the first chamber to a second reaction chamber, the second reaction chamber having an inlet and an outlet and containing a second reactant;
retaining the first reacted assay at the second chamber for a second duration to react with the second reagent to form a second reacted assay.

2. The method of claim 1, wherein accelerating rotation of a flowrotor at a negative angular acceleration further comprises rapidly accelerating rotation of the flowrotor.

3. The method of claim 1, further comprising retaining the assay at the first chamber by terminating the first acceleration while maintaining a first rotational velocity of the flowrotor.

4. The method of claim 1, wherein retaining the assay at the first chamber for a first duration further comprises insitu heating or cooling at the first chamber.

5. The method of claim 1, wherein retaining the assay at the first chamber for a first duration further comprises insitu agitation of the assay by continually reversing the angular acceleration direction.

6. The method of claim 1, wherein the first reagent defines a mixture for amplifying a segment of a DNA in the assay.

7. The method of claim 1, wherein the second reagent is a dye.

8. The method of claim 1, further comprising optically evaluating the final assay.

9. A flowrotor for assay detection, comprising:
a disc having an enclosure formed by an upper surface and a lower surface, the enclosure defining a first channel having an inlet, a reaction chamber and an evaluation chamber connected by a plurality of conduits;
the disc configured to (1) rotate about an axis at a first direction with a first radial acceleration to move an assay from the inlet through a first conduit to a first reaction chamber, (2) retain the assay at the first reaction chamber by maintaining a first angular velocity to induce a reaction with a first reactant and to provide a first reacted assay, (3) accelerate rotation at a negative angular acceleration relative to the first angular velocity until the first angular velocity is substantially reversed thereby moving the first reacted assay from the first reaction chamber to an evaluation chamber.

10. The flowrotor of claim 9, wherein the first reaction chamber further comprises a lip to retain the assay therein.

11. The flowrotor of claim 9, wherein the disc is further configured to retain the assay at the first reaction chamber by terminating the first acceleration while maintaining a first rotational velocity for the flowrotor.

12. The flowrotor of claim 9, wherein the disc is further configured to heat or cool the assay.

13. The flowrotor of claim 9, wherein the disc is further configured to provide insitu agitation of the assay by continually reversing rotation.

14. The flowrotor of claim 9, further comprising a second channel having a second reaction chamber and a second evaluation chamber.

15. The flowrotor of claim 14, wherein the second reaction chamber fluidically communicates with the first reaction chamber.

16. The flowrotor of claim 14, wherein each of the first reaction chamber and the second chamber contains a different reagent.

17. The flowrotor of claim 9, wherein the first reaction chamber comprises at least one of an immobilized lyophilized or dried reagents.

18. A detection system, comprising:
a motor;
a flowrotor; and
a controller in communication with the motor, the controller configured to provide the motor with instructions comprising:
rotate the flowrotor in a first direction with a first radial acceleration to move an assay from the inlet to a first reaction chamber of the flowrotor;
retain the assay at the first reaction chamber by maintaining a first angular velocity to induce a reaction with a first reactant to thereby provide a reacted assay;
accelerate the flowrotor's rotation at a negative angular acceleration relative to the first angular velocity until the first angular velocity is substantially reversed thereby moving the reacted assay from the first reaction chamber to an evaluation chamber.

19. The system of claim 18, wherein the first reaction chamber further comprises an inlet and an outlet and wherein the first radial acceleration delivers the assay to the inlet of the reaction chamber and the negative angular acceleration removes the reacted assay from the outlet of the reaction chamber.

20. The system of claim 18, wherein the first reaction chamber further comprises a lip to retain the assay.

21. The system of claim 18, wherein the flowrotor retains the assay at the first reaction chamber by terminating the first angular acceleration while maintaining a first rotational velocity for the flowrotor.

22. The system of claim 18, wherein the flowrotor is further configured to heat or cool the assay.

23. The system of claim 18, wherein the flowrotor is further configured to provide insitu agitation of the assay by continually reversing angular acceleration direction.

24. The system of claim 18, wherein the flowrotor further comprises a second channel having a second reaction chamber and a second evaluation chamber.

25. The system of claim 24, wherein the second reaction chamber fluidically communicates with the inlet.

26. The system of claim 24, wherein each of the first reaction chamber and the second chamber contains a different reagent.

27. A computer-readable storage device containing a set of instructions to cause a motor to perform a process comprising:
rotate a flowrotor in a first direction with a first radial acceleration to move an assay from the inlet to a first reaction chamber, the first reaction chamber having an inlet and an outlet and containing a first reactant;
retain the assay at the first reaction chamber for a first duration by maintaining a first angular velocity to induce a reaction with the first reactant and provide a reacted assay;
accelerate the flowrotor at a negative angular acceleration relative to the first angular velocity until the angular velocity is reversed thereby moving the reacted assay from the first reaction chamber to a second chamber;
retain the reacted assay at the second chamber for a second duration to induce a reaction with a second reagent to form a final assay; and
rotate the flowrotor at a second radial acceleration to move the final assay to an evaluation chamber.

28. The computer-readable storage device of claim 27, wherein the instructions further cause the motor to perform rapidly accelerate the flowrotor at a negative angular acceleration.

29. The computer-readable storage device of claim 27, wherein the instructions further cause the motor to retain the assay at the first chamber by terminating the first acceleration while maintaining the first rotational velocity of the flowrotor.

30. The computer-readable storage device of claim 27, wherein the instructions further cause a heating or a cooling device to provide insitu heating or cooling to the first chamber.

31. The computer-readable storage device of claim 27, wherein the instructions further cause the motor to agitate the assay by continually reversing the angular acceleration direction.

32. The computer-readable storage device of claim 27, wherein the instructions further cause an optical system to evaluate the final assay.
